(19) United States
(12) Patent Application Publication
(10) Pub. No.: US 2014/0107482 A1
(43) Pub. Date: Apr. 17, 2014

(54) Method and Apparatus for Non-Invasive Treatment of Hypertension Through Ultrasound Renal Denervation

(71) Applicant: Sound Interventions, Inc., (US)

(72) Inventor: Reinhard J. Warnking, East Setauket, NY (US)

(73) Assignee: Sound Interventions, Inc., Stony Brook, NY (US)

(21) Appl. No.: 13/827,301

(22) Filed: Mar. 14, 2013

Related U.S. Application Data
(63) Continuation of application No. 13/503,158, filed on Apr. 20, 2012.
(60) Provisional application No. 61/256,455, filed on Oct. 30, 2009.

(51) Int. Cl. A61N 7/00; A61B 8/14; A61B 8/00

(52) U.S. Cl. CPC A61N 7/00; A61B 8/4494; A61B 8/14
USPC 600/439; 601/2; 600/407

(57) ABSTRACT

Non-invasive inactivation of nerve conduction in a treatment region of a mammalian subject as, for example, a region encompassing a renal artery. A therapeutic ultrasound transducer (31) is engaged with the body of the subject outside of the treatment region, preferably with the skin of the subject in proximity to the treatment region (10). The transducer is actuated to transmit therapeutically effective softly focused ultrasound energy at a level which brings tissues throughout a relatively large impact volume (22), desirably 1 cm³ or larger, to a temperature sufficient to inactivate conduction nerves but insufficient to cause rapid necrosis. The impact volume can be aligned with the treatment region using imaging techniques. The treatment can be applied without imaging or precisely locating individual nerves, and can be used, for example, to inactive renal nerves in treatment of hypertension.

### CROSS-REFERENCE TO RELATED APPLICATIONS

This application claims the benefit of the filing date of U.S. Provisional Patent Application No. 61/256,455, filed Oct. 30, 2009, entitled “METHOD AND APPARATUS FOR NON-INVASIVE TREATMENT OF HYPERTENSION THROUGH ULTRASOUND RENAL DENERVATION,” which is incorporated by reference herein in its entirety. The entire disclosures of U.S. Provisional Patent Application Nos. 61/256,429, filed on Oct. 30, 2009, entitled “METHOD AND APPARATUS FOR TREATMENT OF HYPERTENSION THROUGH ULTRASOUND RENAL DENERVATION,” and 61/292,618, filed on Jan. 6, 2010, entitled “METHOD AND APPARATUS FOR TREATMENT OF HYPERTENSION THROUGH ULTRASOUND RENAL DENERVATION,” are incorporated by reference herein. The entire disclosure of the International Application under the Patent Cooperation Treaty naming Reinhard Warnking as inventor, filed of even date herewith entitled “METHOD AND APPARATUS FOR PERCUTANEOUS TREATMENT OF HYPERTENSION THROUGH RENAL DENERVATION” is also incorporated by reference herein in its entirety.

### TECHNICAL FIELD

The present invention relates to methods and apparatus for inactivation of nerve conduction.

### BACKGROUND OF THE INVENTION

Inactivation of specific nerves associated with a disorder may help treat the disorder. For example, inactivation of renal nerve conduction can be used to treat hypertension. Successful treatment of hypertension is important for many reasons. For example, successful treatment of hypertension has significant clinical benefits in preventing or limiting conditions caused by or exacerbated by hypertension; such as, renal disease, arrhythmias, and congestive heart failure, to name a few. While drug therapy can be used to treat hypertension, it is not always successful. Some people are resistant to drug therapy treatment or experience significant side effects from drug therapy treatment.

Hypertension can be treated by inactivating conduction of the renal nerves surrounding the renal artery. Sympathetic renal nerve activity plays a significant role in the initiation and maintenance of hypertension. When the brain perceives increased renal nerve activity, signaling low blood volume or a drop in blood pressure, it compensates by increasing sympathetic nerve activity to the heart, the liver, and the kidneys, which results in increased cardiac output; insulin resistance; and most importantly, increased renin production by the kidneys. Renin stimulates the production of angiotension, which causes blood vessels to constrict, resulting in increased blood pressure; and stimulates the secretion of aldosterone. Aldosterone causes the kidneys to increase the reabsorption of sodium and water into the blood, increasing blood volume thereby further increasing blood pressure.

It has been established for years that surgically cutting renal nerves results in a decrease in blood pressure and water retention to normal levels, thereby allowing the patients' heart, liver, and kidneys to also return to healthier functioning. It has also been shown that a disruption of the renal nerves has no serious ill effects. However, surgically cutting the renal nerves requires a major surgical procedure. It would be desirable to produce the same result without requiring major surgery.

In order to explain the difficulties associated with accomplishing this task without causing other damage, the anatomy of the renal arteries and nerves will be described now. Shown in FIG. 1 is an illustration of the renal nerves 8 that surround the renal artery 10, which is connected to the kidney 6. The sympathetic renal nerves 8 include both the afferent sensory renal nerves from the kidney 6 to the brain and the efferent sympathetic renal nerves from the brain to the kidney 6. In addition, FIG. 2 shows a cross-section of a renal artery 10. The renal artery wall includes layers: the intima 3, which includes an inner single layer of endothelial cells; the media 5, which is in the center of the artery wall; and the adventitia 4, which is the outside layer. Also shown are the renal nerves 8 that lie within the aventitia 4, on the surface of the renal artery 10, and adjacent to the renal artery 10. As can be seen from these two figures, the renal nerves 8 surround the renal artery 10. Different individuals have the renal nerves 8 in different locations around the renal artery. Thus, the renal nerves may be at different radial distances from the central axis of the renal artery, and also may be at different locations around the circumference of the renal artery. It is not practical to locate the renal nerves by referring to anatomical landmarks. Moreover, it is difficult or impossible to locate individual renal nerves using common imaging technology.

The inability to locate and target the renal nerves 8 makes it difficult to disconnect the sympathetic renal activity using non-surgical techniques without causing damage to the renal artery 10 or causing other side effects. For example, attempts to apply energy to the renal nerves can cause effects such as stenosis, intimal hyperplasia, and necrosis. Other side effects can include thrombosis, platelet aggregation, fibrin clots and vasoconstriction. In addition, the inability to target and locate the renal nerves 8 makes it difficult to ensure that sympathetic renal nerve activity has been disrupted sufficiently to achieve an acceptable therapeutic treatment.

U.S. Pat. No. 7,617,005 suggests the use of a radio frequency (“RF”) emitter connected to a catheter, which is inserted in the renal artery. The RF emitter is placed against the intima and the RF energy is emitted to heat the renal nerves to a temperature that reduces the activity of renal nerves which happen to lie in the immediate vicinity of the emitter. In order to treat all the renal nerves surrounding the renal arteries, the RF emitter source must be repositioned around the inside of each renal artery multiple times. The emitter may miss some of the renal nerves, leading to an incomplete treatment. Moreover, the RF energy source must contact the intima to be able to heat the renal nerves, which may cause damage or necrosis to the single layer endothelium and the intima, potentially causing intimal hyperplasia, renal artery stenosis, and renal artery dissection.

The '005 patent also suggests the use of high-intensity focused ultrasound to deactivate the renal nerves. The described high-intensity focused ultrasound energy source assertedly emits ultrasound energy in a 360° pattern around the axis of the renal artery, and does not need to contact the intima 3. However, the high-intensity focused ultrasound source applies concentrated energy in a thin focal ring surrounding the artery. It is difficult or impossible to align this thin ring with the renal nerves because the renal nerves cannot be visualized and targeted with current technology, and because the renal nerves may lie at different radial distances from the central axis of the renal artery. The latter problem is aggravated in patients who have renal arteries with large variations in shape or thickness. Moreover, the thin focal ring can encompass only a small segment of each renal nerve along the lengthwise direction of the nerves and artery. Since nerves tend to re-grow, a small treatment zone allows the nerves to reconnect in a shorter period of time.

For many years ultrasound has been used to enhance cell repair, stimulate the growth of bone cells, enhance delivery of drugs to specific tissues, and to image tissue within the body. In addition, high-intensity focused ultrasound has been used to heat and ablate tumors and tissue within the body. In high-intensity focused ultrasound, an ultrasonic transducer and associated elements are designed to bring emitted ultrasound waves to a very sharp focus within the body, approximating a theoretical point or line. Thus, the ultrasonic energy applied by the transducer is dissipated within a very small heating volume within the body, on the order of a few mm³. This provides rapid heating of the tissues within such volume to temperatures required for rapid necrosis, typically on the order of 65° C. or more. In some applications, high-intensity focused ultrasound can produce tissue necrosis at a desired point or line without adversely affecting surrounding tissue and intervening structures that the ultrasound energy must pass through. As mentioned above, it is difficult or impossible to use high intensity focused ultrasound to inactive renal nerves because the renal nerves cannot be located using practical non-surgical techniques. This makes it impractical to align the small heating volume with the renal nerves.

### SUMMARY OF THE INVENTION

One aspect of the present invention provides methods for inactivating nerve conduction in a treatment region of a mammalian subject. The method according to this aspect of the present invention desirably includes the step of coupling a therapeutic ultrasound transducer with the body of the subject remote from the treatment region, preferably at the skin of the subject overlying the treatment region. The method preferably further includes the step of actuating the therapeutic ultrasound transducer to transmit therapeutically effective softly focused ultrasound energy into an impact volume of at least about 1.0 cm³. The impact volume desirably encompasses the treatment region of the subject. Most preferably, the therapeutically effective softly focused ultrasound energy is applied throughout the impact volume at a level sufficient to inactivate conduction of nerves, but insufficient to cause tissue necrosis during the time required to inactivate the nerves.

As discussed further below, the impact volume of the softly focused therapeutic ultrasound is many times larger than the focal region used in high intensity focused ultrasound. Because the ultrasonic power is applied throughout the relatively large impact volume at a level appropriate for nerve inactivation, preferred methods according to this aspect of the invention can be performed without locating or targeting individual nerves. All that is required to assure that the nerves in a treatment region of the body are inactivated is to align the impact volume so that it encompasses the treatment region. For example, in treatment of hypertension, the impact volume can be aligned to encompass the renal artery over a portion of its length, without any need to locate or target individual renal nerves. This can be accomplished readily using ultrasonic or other imaging techniques as discussed below.

A further aspect of the invention provides apparatus for inactivating nerve conduction in a treatment region of a mammalian subject. Apparatus according to this aspect of the present invention desirably includes a therapeutic ultrasound transducer adapted to engage with the body of the subject outside of the treatment region as, for example, on the skin of the subject. The apparatus desirably includes an actuator adapted to actuate the therapeutic ultrasound transducer to transmit therapeutically effective softly focused ultrasound energy into an impact volume of at least about 1.0 cm³, wherein the impact volume encompasses the treatment region of the subject and the therapeutically effective softly focused ultrasound energy is at an intensity sufficient to inactivate conduction of nerves throughout the impact volume.

### BRIEF DESCRIPTION OF THE DRAWINGS

FIG. 1 is an anatomical illustration of a renal artery and renal nerves associated with it.

FIG. 2 is a cross-sectional view of a renal artery and renal nerves associated with it.

FIG. 3 is a diagrammatic view depicting apparatus of the according to one embodiment of the present invention engaged with a subject.

FIGS. 4A, 4B, and 4C are diagrammatic view of three different ultrasound transducer assemblies and related elements used in embodiments of the present invention.

FIGS. 5A, 5B, and 5C are diagrammatic views of three different transducers and associated ultrasonic emissions from such transducers.

FIG. 6 is a flowchart of a method according to one embodiment of the present invention.

FIG. 7 is a flowchart of a method according to a further embodiment of the present invention.

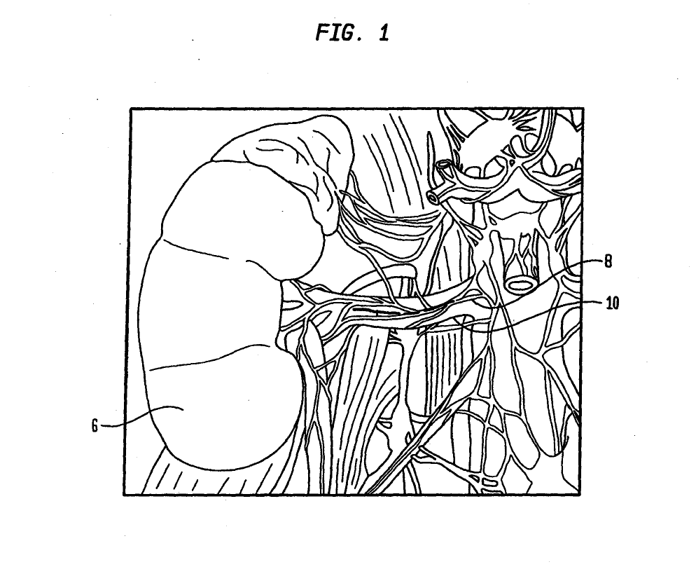

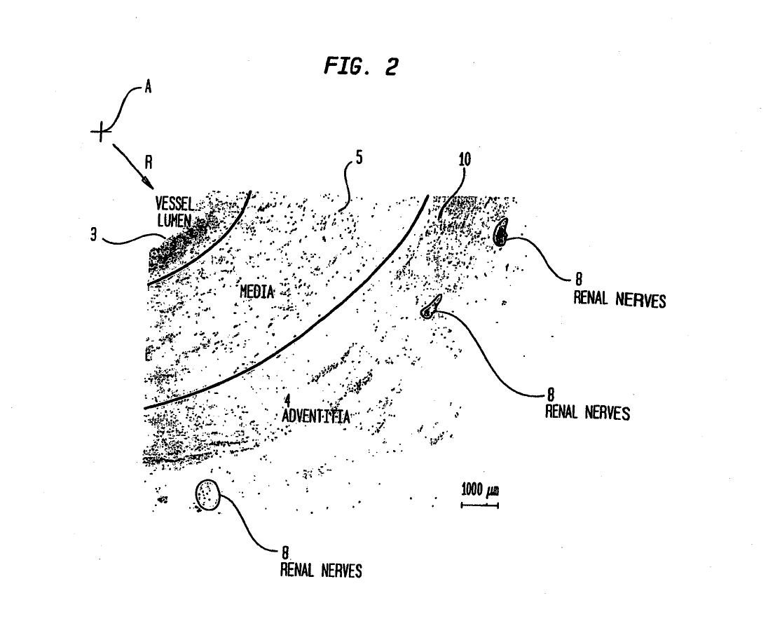

### DETAILED DESCRIPTION

Apparatus and methods according to certain embodiments of the present invention can be used to non-invasively inactivate nerve conduction. For example, the apparatus and methods can be used to inactivate conduction of all the renal nerves 8 that surround the renal artery 10. This includes renal nerves 8 which are located in, on the surface of, and adjacent to the renal artery 10. Such inactivation can be achieved without surgery and thus without typical risks, such as thrombosis, infection, and other collateral damage.

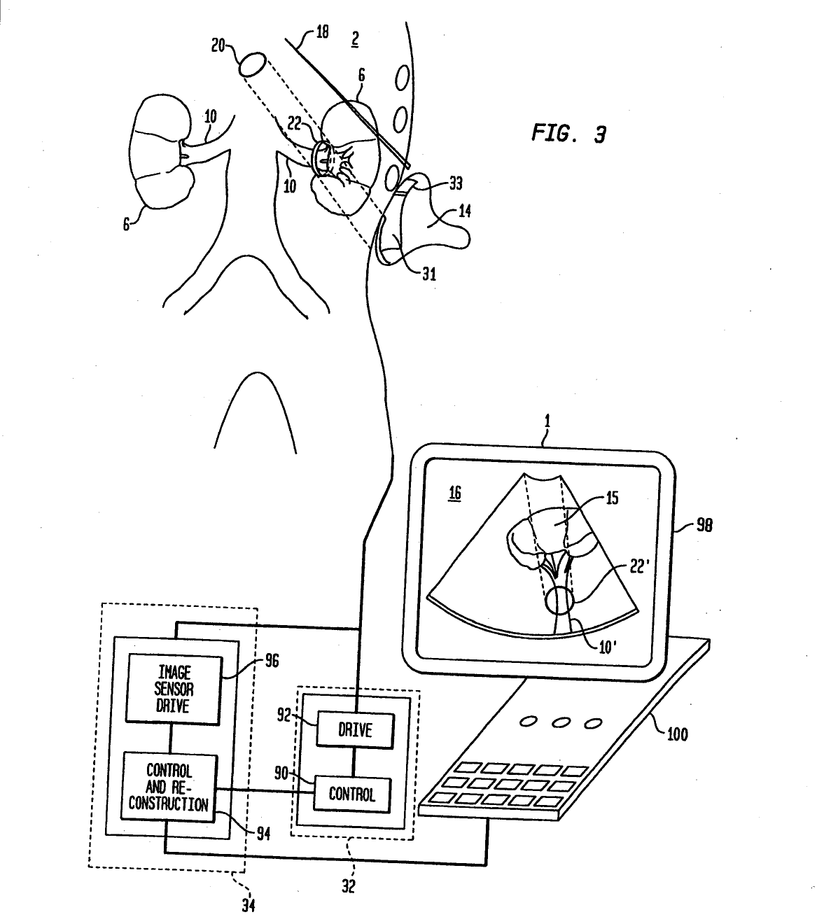

Apparatus 1 according to one embodiment of the present invention (FIG. 3) includes an ultrasound transducer assembly 14 and an ultrasound system 32, also referred to herein as an actuator. The actuator 32 incorporates a control computer 90 linked to a driver 92 adapted to generate electrical signals at the desired ultrasonic frequency as commanded by the control computer 92. The ultrasound transducer assembly 14 in this embodiment includes a therapeutic ultrasound transducer 31 and an imaging transducer 33 mechanically connected to the therapeutic transducer. In the particular embodiment of FIG. 3, the imaging transducer lies at a fixed position and orientation relative to the therapeutic transducer, and the therapeutic transducer has a fixed focal length. Although these transducers are depicted as separate elements, they may be integrated as discussed below. In the particular procedure depicted in FIG. 3, the transducer assembly is located extra-corporeally to the subject 2 and engages with the skin of the subject 2. This is typically performed using a coupling gel on the skin of the subject 2.

The imaging transducer 33 forms a part of an imaging unit or “imager.” The imager further includes an imaging subsystem 34 which incorporates a control and reconstruction computer 94 linked to an image transducer driver and sensor 96, which in turn is linked to the imaging transducer 33. Driver and sensor 96 is arranged to actuate the imaging transducer to emit ultrasonic imaging signals, to receive electrical signals generated by the imaging transducer responsive to ultrasonic echoes reflected by the subject, and to transfer the information in the electrical signals to the control and reconstruction computer 94. The control and reconstruction computer 94 is arranged to control the driver and sensor unit and to reconstruct an image of the subject's tissues from the electrical signals received through driver and sensor 96. The control and reconstruction computer 94 is linked to a display 98, as well as to the control computer 90 of the actuator. Control computer 92 of the actuator and control and reconstruction computer 96 of the imager are linked to user input controls 100 for receipt of user commands. Although elements 90-96 are shown as separate functional elements, these can be integrated with one another. The algorithms required for control of an imaging transducer and reconstruction of an image are well-known in the art.

The aperture of the therapeutic transducer 31 is selected to be large enough to avoid skin burn. As further discussed below, the therapeutic transducer supplies ultrasonic emissions having sufficient total power to heat tissues within an impact volume 22 inside the patient's body. Transmission of ultrasound through the skin typically results in some dissipation of energy within the skin, and thus heating of the skin. This limits the power which can be transmitted through a given area of the skin without causing burns. Therefore, it is normally necessary to apply the therapeutic ultrasound over an area of the skin larger than the cross-sectional area of the impact volume in a plane perpendicular to the direction of propagation of the ultrasonic energy. The size of the emitting aperture of the therapeutic transducer controls the area of the skin used to transmit the ultrasonic energy into the body.

When inactivating renal nerve conduction, the ultrasound transducer assembly 14 is preferably positioned on the back of the subject 2 near the kidney 6 to provide a relatively large coupling window with little intervening tissue and, typically, no intervening bones or other obstacles which are highly reflective to ultrasound. The large coupling window will further permit a large aperture therapeutic transducer 31 to be utilized. In the preferred embodiment, the typical size of the aperture is about 20 cm², however this size will change depending on the treatment region and the particular body structure of the subject 2.

In a method according to one embodiment of the present invention, the computer 94 and driver 96 actuate imaging transducer 33 to transmit an ultrasound imaging signal 18, which is reflected off structures of the subject 2 to produce echoes. The echoes are received by the imaging transducer 33 and converted to electrical signals, which in turn are used by computer 94 to generate an image 16 of a body region on display 98 that may be viewed by a user. In a preferred embodiment, the image 16 includes a graphic overlay 15, which shows the anticipated energy path of the therapeutic ultrasound energy and the location of the impact volume 22 where the ultrasonic energy emitted by the therapeutic transducer converges to the intensity required for nerve deactivation. Because the therapeutic transducer 31 has a fixed focal length and is in a fixed spatial relationship with the imaging transducer 33, the locations of the path and impact volume in the frame of reference of the imaging transducer and image 16 are known, so that the overlay can be displayed.

A user preferably looks at the graphic overlay 15 to adjust the ultrasound transducer assembly 14 so that the depiction 22′ of the impact volume encompasses the image 10′ of treatment region 10 (shown as the renal artery) and the energy path is not obstructed by bone or air. Once the impact volume 22 encompasses the treatment region 10, the user instructs control computer 90 to actuate therapeutic transducer 31, whereupon the therapeutic transducer emits the therapeutically effective softly focused ultrasound energy 20 to the impact volume 22. The therapeutic energy 20 brings the impact volume to a temperature as discussed below and thus inactivates conduction of all the nerves in the impact volume 22. It is not necessary to image or locate individual nerves.

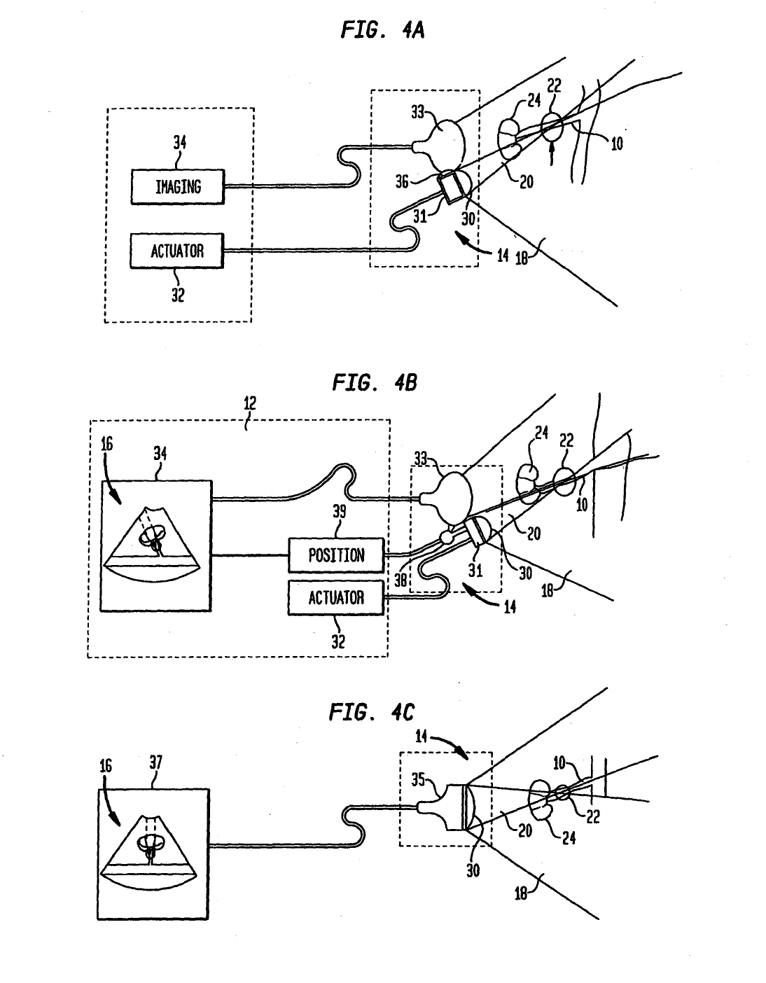

FIG. 4A depicts the ultrasound transducer assembly 14 of FIG. 3, including imaging transducer 33 and therapeutic transducer 31. The diagnostic imaging transducer 33 is connected to the imaging subsystem 34, while the therapeutic sub-assembly 31 is connected to actuator 32. The imaging transducer 33 emits and receives imaging ultrasound 18 and imaging subsystem 34 produces the image, whereas the therapeutic transducer 31 transmits therapeutically effective softly focused ultrasound energy 20 to the treatment region. In this embodiment, the therapeutic transducer 31 is mechanically fixed by a fixed link 36 to the imaging transducer 33 at an angle that allows the impact volume of the therapeutic ultrasound energy to be located within the imaged body region.

Referring to FIG. 4B, another embodiment of the ultrasound transducer assembly 14 also includes an imaging transducer 33, which emits imaging ultrasound 18, and therapeutic transducer 31. However, the mechanical connection 38 between the two transducers is not fixed. The mechanical connection 38 includes a position sensor 39, which transmits information about the position of the therapeutic transducer 31 relative to the imaging transducer 33 to the imaging subsystem 34 (FIG. 3). The control and reconstruction computer uses such position information to transform the position of the therapeutic transducer 31 into the frame of reference of the imaging transducer, or vice-versa, so that the overlay of the impact volume and path can be accurately displayed on the image 16 of the subject's body. Techniques for mathematical transformation of images between frames of reference are well-known in the art.

Referring to FIG. 4C, the ultrasound transducer assembly 14 may also be a phased array transducer 35 or similarly an annular array transducer (not shown). Both of these transducers have separate transducer elements that may be activated separately, as known to one skilled in the art. In one embodiment, the phased array transducer 35 performs both the imaging, using imaging ultrasound 18, and the transmission of the therapeutically effective softly focused ultrasound energy 20. The phased array is connected to a system 37 which incorporates the elements of imager subsystem 34 and actuator 32 (FIG. 3). This combined system 37 is arranged to both generate the image 16 using transducer 35 and to control the plurality of transducer elements 40 of the ultrasound transducer array 35, to generate the therapeutically effective softly focused ultrasound energy 20. When generating the image 16, the computer of system causes at least one and up to several hundred transducer elements 40 to receive the reflected echoes. This embodiment advantageously reduces the risk of incorrectly identifying the position of the treatment region 10 because diagnostic as well as therapeutic pathways of the ultrasound energy 20 are identical.

Typically, the transducer assembly 14 is provided as a replaceable unit which can be mated with a reusable device including the actuator 32 and imaging subsystem 34 (FIG. 3). The transducer assembly desirably includes a data carrying element such as a bar code, electronic memory or the like, and the reusable device is equipped to read the data on such element and convey the same to the computers of the actuator and imaging subsystem. The data carried on the transducer assembly includes parameters of the transducers, such as the proper operating frequency for the therapeutic and imaging transducers, the focal length of the therapeutic transducer and the size and shape of the emitting aperture of the therapeutic transducer. Alternatively, the data carried on the transducer assembly may include identifying information such as a serial number which can be used by the computers of the actuator and imaging subsystem to retrieve information pertaining to the particular transducer assembly from a central database accessible through a communications link such as the internet.

A deformable coupling medium 30 (FIGS. 4A-4C) may be provided between the therapeutic transducer 31 or 35 and the subject. The deformable coupling may include a material that allows the therapeutic ultrasound energy 20 to be transmitted through it. For example, the deformable coupling medium may include a flexible or elastic bag filled with water or a gel. By applying a force on the ultrasound transducer to compress or decompress the deformable medium, the location of the impact volume 22 of the therapeutically effective softly focused ultrasound energy 20 may be adjusted to encompass the treatment region 10.

In another embodiment, the therapeutic transducer may be connected to a mechanical system arranged to move the therapeutic transducer. The control and reconstruction computer of the imaging subsystem may be arranged to compare the location of the impact volume with the location of the treatment region and to actuate the mechanical system to move the therapeutic transducer position as required to assure that the location of the impact volume 22 encompasses the treatment region 10. In such a system, the user may designate the boundaries of the treatment region in the frame of reference of the image, such as by providing manual inputs to the computer to move a cursor displayed on the image to the boundaries of the treatment region and entering inputs indicating that the cursor is on the boundary.

In other embodiments, the imager uses image acquisition elements which are not associated with the therapeutic transducer. Merely by way of example, imaging modalities such as X-ray, CAT, MRI, and the like can be used. Provided that the position of the therapeutic transducer can be determined in the frame of reference of the imaging system, or in another frame of reference having a known transformation to the frame of reference of the imaging system, the location of the impact volume and the image of the subject's body can be brought into a common frame of reference.

In the embodiments discussed above, the therapeutic transducer focuses the ultrasound energy 20, but only to a degree. As used in this disclosure, the with respect to ultrasonic energy, the term “focus” means that the intensity of the ultrasonic energy increases in the direction of propagation away from the emitter to a location remote from the emitter where the intensity is at a maximum. In conventional high-intensity focused ultrasound, the transducer is designed and operated to focus the energy into a focal region such as a point or line which has volume as close to zero as possible, typically a few mm³. The ultrasonic energy has high intensity within this small focal region, but the intensity diminishes as sharply as possible at the boundaries of the focal region. By contrast, in the preferred embodiments of the present invention, the therapeutic transducer is constructed and operated so that the focal region is intentionally blurred and the ultrasonic energy has reasonably uniform intensity throughout a relatively large region, referred to herein as the “impact volume” surrounding the point of maximum intensity. The intensity within the impact volume desirably is uniform enough to produce the desired therapeutic effect throughout the impact volume. In the preferred embodiments of the present invention, the desired therapeutic effect is inactivation of nerve conduction without ablation or necrosis of tissue. As discussed below, this typically requires heating solid tissues to between about 42° C. but less than 65° C. as discussed below. Thus, the intensity of the ultrasonic energy in the impact volume should be uniform enough to heat substantially all solid tissues within the impact volume, other than blood and those which are in intimate contact with a cooling medium such as blood, to 42-65° C., but no tissues are heated to above 65° C. The impact volume preferably has a volume of 1 cm³, but less than 5 cm³. Stated another way, the ultrasonic energy is still focused, in that it increases in intensity in the direction of propagation from the transducer to the impact volume, but the focus is a soft focus. The preferred soft focus is different from the prior art devices that use high-intensity sharply focused ultrasound for ablating tumors and other tissue because the impact volume of the softly focused ultrasound is 10 to 100 times larger than the volume of focal region in high-intensity sharply focused ultrasound. In addition, because the ultrasound energy 20 is softly focused, the maximum intensity of the ultrasound energy in the impact volume is 10 to 100 times less than the maximum intensity of high-intensity sharply focused ultrasound used in ablation of tissue. For example, in the softly focused ultrasound, the maximum intensity in the impact volume, which is also the maximum intensity in the beam path, typically is about 1 Watt/cm² or less to about 10 Watt/cm².

As can be seen in FIGS. 4A, B, and C, the softly focused ultrasound energy 20 is directed to the treatment region, which in FIGS. 4A, B, and C is the renal artery 10, so that the impact volume 22 will encompass the renal artery 10 and the nerves within the adventitia of the renal artery and surrounding the adventitia. In regions along the path of propagation of the ultrasound before and beyond the impact volume 22, the intensity of the ultrasound energy 20 is too weak to inactivate nerve conduction or cause tissue damage. Within the impact volume, the intensity of the ultrasound energy 20 is therapeutically effective in that it is strong enough to inactivate nerve conduction, but it is not strong enough to ablate tissue or cause necrosis in the time required for nerve inactivation. Research shows that nerve damage occurs at much lower temperatures and much faster than tissue necrosis. See Bunch, Jared. T et al. “Mechanisms of Phrenic Nerve Injury During Radiofrequency Ablation at the Pulmonary Vein Orifice, Journal of Cardiovascular Electrophysiology, Volume 16, Issue 12, Pg. 1318-1325 (Dec. 8, 2005), incorporated by reference herein. When applying the therapeutically effective softly focused ultrasound energy 20 to inactivate renal nerve 8 conduction, as shown in FIG. 3 and FIG. 4, the ultrasound energy 20 is strong enough to inactivate the renal nerve 8 conduction yet not strong enough to cause damage, such as, stenosis, intimal hyperplasia, intimal necrosis, or other injuries that would require intervention.

Since necrosis of tissue typically occurs at temperatures of 65° C. or higher for about 10 sec or longer while inactivation of renal nerve conduction typically occurs when the renal nerves are at temperatures of 42° C. or higher for several seconds or longer, the dosage of the ultrasound energy is chosen to keep the temperature in the impact volume 11 within this temperature range for several seconds or longer.

The therapeutic transducer is designed to operate, for example, at a frequency of about 1 MHz to about a few tens of MHz, and typically at about 5 MHz. To generate the therapeutic dosage of ultrasound energy within the impact volume, the acoustic power emitted by the transducer in the preferred embodiments typically is about 10 to about 100 watts. The duration of the power application typically is about 10 seconds to about 30 seconds, but may be from about 5 seconds to about a minute or more. The precise power level and duration to provide the correct dosage can be determined for each treatment region by mathematical modeling and, preferably, by preclinical testing to evaluate actual temperatures achieved with different dosages. Such preclinical testing is helpful due to the complexity of the biological structure such as tissue layers and physical dynamics such as blood flow.

Moreover, the transmission of the therapeutically effective softly focused ultrasound energy 20 may be as a pulsed function with a duty cycle synchronized and interlaced with the imaging ultrasound duty cycles. The pulsed operation allows the apparatus 1 to generate the image and the therapeutic ultrasound in real-time without obscuring the image with the therapeutic ultrasound.

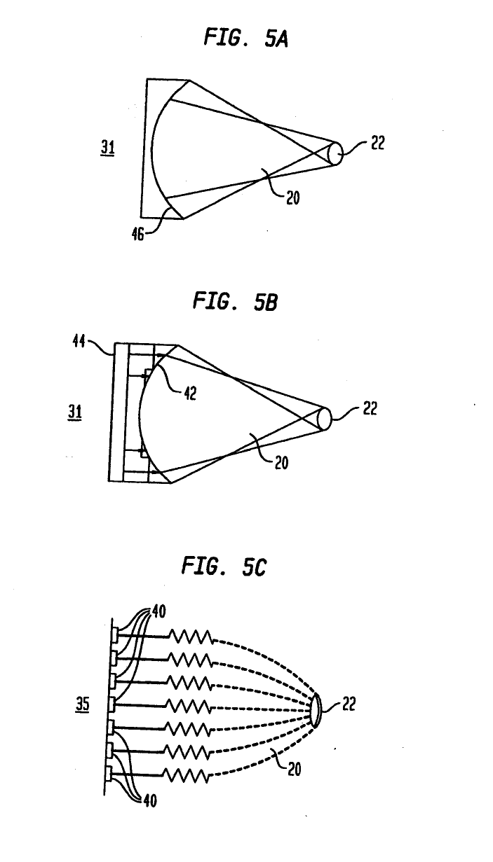

As shown in FIG. 5A, the therapeutic transducer 31 may be geometrically formed to provide the therapeutically effective softly focused ultrasound energy. Rather than a partial spherical shape, which would produce a sharply focused region, the emitting surface 46 of the transducer is a non-spherical shape, for example, a partial ellipsoid. The ellipsoid causes the ultrasound energy to converge but not to a single point. Mathematical techniques for determining the intensity distribution resulting from a particular emitting surface shape are well known in the art, and can be used to select the correct shape for a soft-focus transducer. The shape and size of the non-spherical transducer is selected to generate an impact volume that is at least 1 cm³.

In another embodiment, shown in FIG. 5B, the therapeutic transducer 31 includes a planar emitter 44 which transmits unfocused ultrasound energy and an ultrasonic lens, such as a Fresnel lens 42, which provides the focusing action to form the unfocused ultrasound energy into be therapeutically effective softly focused ultrasound energy 20. In order to accomplish this, the configuration of the lens deviates slightly from the conventional configuration used to provide a sharp point focus. For example, a conventional sharp-focus lens has a partially spherical surface or, in the case of a Fresnel lens, concentric rings configured to simulate a spherical surface. To provide soft-focused ultrasound, the surface of lens 42 deviates slightly from this configuration. Here again, mathematical techniques for ultrasonic lens design are well known. Lens 42 may be replaceable by the user, so that the user can alter the location of the impact volume by selecting a different lens based on the difference between the location of the graphic overlay impact volume and the location of the treatment as displayed on the imaging system. Each replaceable lens 42 may have a different focal length to allow the location of the impact volume 22 of the therapeutically effective softly focused ultrasound energy to be adjusted to encompass the treatment region 10. Individual lenses may bear machine-readable information which can be read by the actuator and/or imaging subsystem as, for example, the focal length of the lens.

Where the therapeutic ultrasound transducer includes a phased array 35 (FIG. 5C) the actuator operates the individual transducer elements 40 of the phased array 35 to transmit ultrasound energy 20 in a timed sequence to provide the therapeutically effective softly focused ultrasound energy 20. In conventional operation to yield a sharp focus, the time sequence is selected so that emissions from elements closer to the focal point are delayed relative to emissions from elements further from the focal point. Thus, the ultrasonic energy from all of the transducer elements arrives at the focal point exactly in phase. To provide a softly focused beam, the delay times are varied slightly from those used to provide a sharp focus. The actuation of the phased array may also include actuation of different elements at different amplitudes. Here again, mathematical techniques for determining the effect of a given pattern of delay times and actuation amplitudes are well known. The phased array 35 may contain hundreds of transducer elements 40.

The pattern of actuation of the plurality of transducer elements 40 in can be varied to move the location of the impact volume of the therapeutic energy 20 to be adjusted to encompass the treatment region. For example, a user may identify a treatment region and an ultrasound energy path on the diagnostic image of the body region, which may be displayed by the computer systems discussed above, and the computer system may determine the activation sequence and a transducer element power output for each transducer element 40 based on the identified treatment region and the identified ultrasound energy path. Furthermore, the pattern of actuation may also be adjusted based on the on the subject's body structures. In this embodiment, certain elements 40 the acoustic power output of the various elements is adjusted so that the ultrasound energy 20 is lower at certain points in the energy path where structures such as bones may be obstructing the therapeutic ultrasound energy's path to the treatment region. This adjustment may include, for example, reducing the power to some elements, entirely deactivating some elements, or both.

A flowchart of a method according to one embodiment of the present invention is shown in FIG. 6. The method of FIG. 6 uses a transducer assembly incorporating separate therapeutic and imaging transducers. The method includes the step of engaging the ultrasound transducer assembly with the skin of the subject (Step 56) and controlling the therapeutic transducer, through the actuator, to transmit therapeutically effective ultrasound energy to the impact volume (Step 66). The method optionally may include numerous additional steps, which are shown in dashed lines to indicate that they are optional. First the user connects the ultrasound transducer assembly to the actuator and imaging subsystem (Step 50). The actuator and imaging subsystem read information from the transducer assembly, and determine the focal length and size of the aperture of the therapeutic transducer and the proper actuation frequencies for the imaging and the therapeutic transducers (Step 52). The control computer determines the correct actuation amplitudes to provide the desired dosage of the therapeutic energy based on the aperture and the frequency (Step 54). This may be accomplished, for example by reading dosage information from the transducer assembly or by reading a value from a look up table programmed during manufacture of the transducer or by calculating the value based on the parameters read from the transducer.

Next, the user engages the transducer assembly with the skin of the subject (Step 56). This is typically accomplished using a deformable coupling medium such as coupling gel on the skin of the subject. The imager will then display an image of a part of the subject's body with the propagation path of the ultrasonic energy and location of the impact volume overlaid on the image (Step 58). The user adjusts the position of the therapeutic transducer (Step 60), while looking at the graphic display of the image to determine if the energy path is obstructed by bone or air (Step 62) and while looking to see that the impact volume encompasses the treatment region (Step 64). Where the transducer assembly includes an adjustable coupling between the therapeutic transducer and imaging transducer, the user may adjust the coupling in this process. The user may continue to move the transducer assembly until a position is found where there are no obstructions and the impact volume encompasses the treatment region. As the user adjusts the location of the therapeutic transducer, the deformable coupling medium attached to the therapeutic transducer may be compressed or decompressed. When the user determines that the impact volume is positioned correctly, the user initiates the transmission of the therapeutically effective softly focused ultrasound energy (Step 66). It should be noted that there is no need for the user to locate individual nerves in the treatment region. Rather, the user need only align the impact volume with the treatment region and actuate the transducer in order to achieve inactivation of nerves within the treatment region.

If the user cannot position the therapeutic transducer so that there are no obstructions in the path of propagation, the user can select a different transducer assembly with a smaller or differently-shaped aperture (Step 68) and return to the beginning of the process (Step 50). Where the therapeutic transducer includes a replaceable lens, the user may change the lens on the therapeutic sub-assembly (Step 72). When the lens is changed, the actuator or imaging subsystem reads information from the lens to re-determine the focal length and recalculate the proper settings to provide the desired dosage of therapeutic ultrasound energy, and the rest of the process proceeds from step 54.

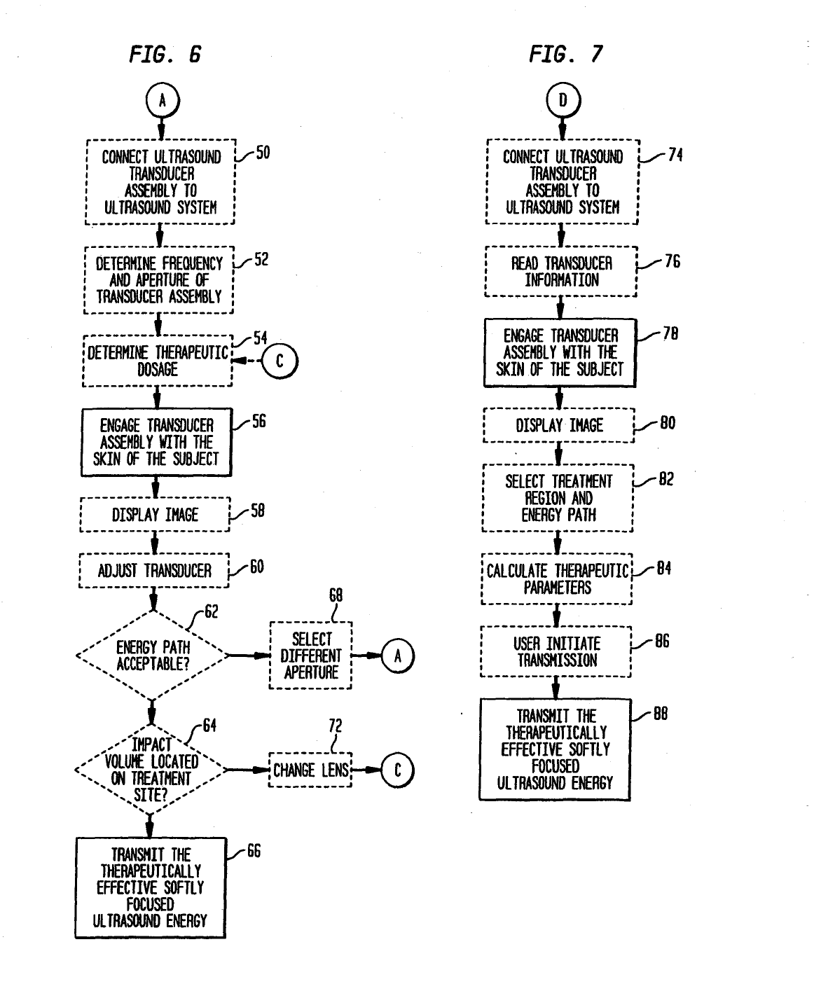

A method according to an embodiment using a transducer assembly incorporating a single phased array transducer with a plurality of transducer elements is depicted in FIG. 7. In FIG. 7 as well, many of the steps are optional. Here again, the user first connects the ultrasound transducer assembly to the actuator and imaging subsystem (Step 74). Here again, the actuator and imaging subsystem reads the transducer information from the transducer assembly (Step 76). The user then engages the transducer assembly with the skin of the subject (Step 78) and the imaging subsystem uses elements of the phased array to transmit an imaging ultrasound signal and receive the resulting echoes. The imaging subsystem displays the image of the body region to the user (Step 80). The user operates the system to bring the impact volume to a desired location encompassing the treatment region and provide a propagation path free of obstructions (Step 82). The user may physically move the phased array to move the impact volume, or may actuate the control computer of the actuator to select different parameters for operation of the array, so as to move the impact volume to a different location relative to the array. The computer system in the actuator calculates the therapeutic parameters to be applied to the phased array such (Step 84). In this step, a timing sequence and a power level is calculated for each of the plurality of transducer elements 40 to produce the therapeutically effective softly focused ultrasound energy at the specified impact volume location. The user then inputs a signal to initiate the transmission of the therapeutic ultrasound (Step 86). In response to that signal, the computer system controls the plurality of transducer elements (Step 88), to transmit the softly focused ultrasound energy to the impact volume. Here again, the therapeutic ultrasound may also be generated in a pulsed mode synchronized and interlaced with the diagnostic imaging sequence to allow a real time display of the image during treatment.

Numerous other variations and combinations of the features discussed above can be utilized without departing from the present invention as defined by the claims. As noted above, imaging may be accomplished using modalities other than ultrasound imaging. Also, a separate imaging transducer may be coupled with a phased array transducer. In this variation the phased array transducer would be used solely for transmitting the therapeutically effective softly focused ultrasound energy. Transducers having emitting surfaces other than an ellipsoid, and lenses other than Fresnel lenses can be used provide the blurring or soft focus effect. Further, lenses can be used with non-planar transducers.

The subject may be a human or non-human mammalian subject.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for inactivating nerve conduction in a treatment region of a mammalian subject comprising the steps of:
(a) coupling a therapeutic ultrasound transducer with the body of the subject remote from the treatment region; and
(b) actuating the therapeutic ultrasound transducer to transmit therapeutically effective softly focused ultrasound energy into an impact volume of at least about 1.0 cm³, wherein the impact volume encompasses the treatment region of the subject and wherein the therapeutically effective softly focused ultrasound energy is applied throughout the impact volume at a level sufficient to inactivate conduction of nerves.

2. The method of claim 1 wherein the step of coupling the transducer to the body of the subject is performed by coupling the transducer to the skin of the subject.

3. The method of claim 2, wherein the treatment region of the subject encompasses the subject's renal artery.

4. The method of claim 3, further comprising the steps of:
(a) acquiring an image of a portion of the subject's body including the treatment region in a common frame of reference with the transducer;
(b) displaying a representation of the impact volume overlaid on the acquired image; and
(c) adjusting the transducer based on the displayed representation and image so as to position the impact volume to encompass the treatment region before actuating the transducer to transmit the therapeutically effective ultrasound energy.

5. (canceled)

6. (canceled)

7. (canceled)

8. The method of claim 4, wherein the ultrasound transducer assembly comprises the therapeutic ultrasound transducer and an imaging sub-assembly, the therapeutic transducer being mechanically coupled to the imaging sub-assembly at an angle that allows the impact volume of the therapeutically effective softly focused ultrasound energy to be within the imaged body region, and wherein the step of generating an image includes actuating the imaging subassembly to transmit an imaging ultrasound and receive echoes.

9. (canceled)

10. (canceled)

11. The method of claim 1, wherein the therapeutic sub-assembly is geometrically formed to provide softly focused ultrasound energy.

12. The method of claim 4, wherein the step of adjusting the therapeutic ultrasound transducer includes changing a replaceable lens associated with the therapeutic ultrasound transducer.

13. The method of claim 4, wherein the therapeutic ultrasound transducer comprises a phased array transducer and the step of acquiring an image includes actuating the phased array transducer to transmit imaging ultrasound and receive echoes.

14. (canceled)

15. (canceled)

16. The method of claim 1, wherein the therapeutic ultrasound transducer is actuated to emit at an acoustic power level of about 10 to about 100 Watts for about 10 to about 30 seconds.

17. The method of claim 1, wherein the transmission of the therapeutically effective softly focused ultrasound energy causes the temperature of the solid tissues within the impact volume to rise above 42° C. without heating any part of the treatment region to 65° C. or more.

18. The method of claim 1, wherein the therapeutically effective softly focused ultrasound energy is transmitted in a pulsed function synchronized and interlaced with imaging ultrasound signals.

19. An apparatus for inactivating nerve conduction in a treatment region of a mammalian subject comprising:
(a) a therapeutic ultrasound transducer adapted to engage with the body of the subject outside of the treatment region; and
(b) an actuator adapted to actuate the therapeutic ultrasound transducer to transmit therapeutically effective softly focused ultrasound energy into an impact volume of at least about 1.0 cm³, wherein the impact volume encompasses the treatment region of the subject and the therapeutically effective softly focused ultrasound energy is at an intensity sufficient to inactivate conduction of nerves throughout the impact volume.

20. The apparatus of claim 19, wherein the therapeutic ultrasound transducer is adapted to engage the skin of the subject.

21. The apparatus of claim 20, wherein therapeutic transducer is adapted to engage the skin of the subject at a adjacent the kidneys of the subject so that the impact volume encompasses a renal artery of the subject.

22. The apparatus of claim 19, further including an imager adapted to acquire an image of a portion of the body of the subject including the treatment region in a common frame of reference with the therapeutic ultrasound transducer and a display adapted to display the acquired image with a representation of the impact volume overlaid thereon.

23. (canceled)

24. (canceled)

25. (canceled)

26. The apparatus of claim 19, wherein the therapeutic transducer is geometrically formed to provide softly focused ultrasound energy.

27. The apparatus of claim 19, wherein the therapeutic transducer further comprises a replaceable ultrasonic lens.

28. (canceled)

29. (canceled)

30. (canceled)

31. The apparatus of claim 19, wherein the actuator is operative to control the therapeutic ultrasound transducer so that to transmit therapeutically effective softly focused ultrasound energy at an acoustic power level of about 10 to about 100 Watts for about 10 to about 30 seconds.

32. The apparatus of claim 19, wherein the actuator is operative to control the therapeutic ultrasound transducer so that the therapeutically effective softly focused ultrasound energy causes the temperature of the treatment region to be less than 65° C. but above 42° C.

33. (canceled)

34. Apparatus for inactivating nerve conduction in a treatment region of a mammalian subject comprising the steps of:
(a) a therapeutic ultrasound transducer;
(b) means for coupling the therapeutic ultrasound transducer with the body of the subject remote from the treatment region; and
(c) means for actuating the therapeutic ultrasound transducer to transmit therapeutically effective softly focused ultrasound energy into an impact volume of at least about 1.0 cm³, wherein the impact volume encompasses the treatment region of the subject and wherein the therapeutically effective softly focused ultrasound energy is applied throughout the impact volume at a level sufficient to inactivate conduction of nerves.
